(19) United States
(12) Patent Application Publication
(10) Pub. No.: US 2016/0051527 A1
(43) Pub. Date: Feb. 25, 2016

(54) METHODS AND KITS FOR TREATING AND CLASSIFYING INDIVIDUALS AT RISK OF OR SUFFERING FROM A NEUROLOGICAL DYSFUNCTION OR DISORDER

(71) Applicant: COURTAGEN LIFE SCIENCES, INC., Woburn, MA (US)

(72) Inventor: Richard Boles, Woburn, MA (US)

(21) Appl. No.: 14/762,446

(22) PCT Filed: Jan. 22, 2014
(86) PCT No.: PCT/US14/12494

§ 371 Date: Jul. 21, 2015

Related U.S. Application Data
(60) Provisional application No. 61/778,217, filed on Mar. 12, 2013.
(60) Provisional application No. 61/755,226, filed on Jan. 22, 2013.

(51) Int. Cl. A61K 31/445

(52) U.S. Cl. CPC A61K 31/445

(57) ABSTRACT

The present disclosure provides methods and kits for treating and classifying individuals at risk of or suffering from a neurological dysfunction or disorder. In general, the individuals are treated and/or classified based on the presence of a loss-of-function mutation in nuclear DNA that encodes choline O-acetyltransferase (ChAT). Treatment involves the administration of a therapeutically effective amount of an acetylcholinesterase (ACNE) inhibitor.

### BACKGROUND

Neurological dysfunctions and disorders continue to be a major health threat in the population. Neurological dysfunctions and disorders occur due to dysfunction of the neurons in the central nervous system as well as the peripheral nervous system.

One frequent contributing factor of neurological dysfunctions and disorders is mitochondrial disease. Some mitochondrial diseases are due to mutations or deletions in the mitochondrial genome. Mitochondria divide and proliferate with a faster turnover rate than their host cells, and their replication is under control of the nuclear genome. If a threshold proportion of mitochondria in a cell is defective, and if a threshold proportion of such cells within a tissue have defective mitochondria, symptoms of tissue or organ dysfunction can result. Practically any tissue can be affected, and a large variety of symptoms may be present, depending on the extent to which different tissues are involved.

### SUMMARY

The present invention relates to methods and kits for treating and classifying individuals at risk of or suffering from a neurological dysfunction or disorder, and in particular, those neurological dysfunctions or disorders associated with loss of function mutations in choline O-acetyltransferase (ChAT). In some embodiments neurological dysfunctions or disorders associated with loss of function mutations in ChAT are treated with an acetylcholinesterase (AChE) inhibitor.

In certain embodiments, the present invention provides methods of treating an individual at risk of or suffering from a neurological dysfunction or disorder, the method comprising administering to the individual a therapeutically effective amount of an AChE inhibitor, wherein nuclear DNA of the individual that encodes ChAT includes a loss-of-function mutation.

In certain embodiments, the present invention provides methods of treating an individual at risk of or suffering from a neurological dysfunction or disorder, the method comprising administering to the individual a therapeutically effective amount of an AChE inhibitor, wherein, prior to administration, the individual has been determined to possess a loss-of-function mutation in nuclear DNA that encodes ChAT.

In certain embodiments, the present invention provides methods of treating an individual at risk of or suffering from a neurological dysfunction or disorder, the method comprising determining that the individual possesses a loss-of-function mutation in nuclear DNA that encodes ChAT and administering to the individual a therapeutically effective amount of an AChE inhibitor.

In certain embodiments, the present invention provides methods of aiding in the selection of a therapy for an individual at risk of or suffering from a neurological dysfunction or disorder, the method comprising obtaining a sample of nuclear DNA from the individual, processing the sample to determine whether the individual possesses a loss-of-function mutation in nuclear DNA that encodes ChAT and classifying the individual as one that could benefit from therapy with an AChE inhibitor if the step of processing determines that the individual possesses a loss-of-function mutation in nuclear DNA that encodes ChAT. In some embodiments, processing comprises sequencing at least a portion of nuclear DNA that encodes ChAT. In some embodiments, the methods further comprise administering to the individual a therapeutically effective amount of an AChE inhibitor.

In certain embodiments, the present invention provides methods of classifying an individual at risk of or suffering from a neurological dysfunction or disorder, the method comprising obtaining a sample of nuclear DNA from the individual, processing the sample to determine whether the individual possesses a mutation in nuclear DNA that encodes ChAT, and classifying the individual as one that does or does not possess a mutation in nuclear DNA that encodes ChAT. In some embodiments, processing comprises sequencing at least a portion of nuclear DNA that encodes ChAT. In some embodiments, the mutation is a loss-of-function mutation. In some embodiments, the methods further comprise providing the individual or a physician treating the individual with information regarding the mutation. In some embodiments, the information references a correlation between the mutation and the potential benefits of therapy with an AChE inhibitor.

In certain embodiments, the present invention provides kits for classifying an individual at risk of or suffering from a neurological dysfunction or disorder, the kit comprising primers for amplifying a target region of nuclear DNA that encompasses part or all of the codon for amino acids 340 and/or 510 of a ChAT gene product. In certain embodiments, the present disclosure provides kits for classifying an individual at risk of or suffering from a neurological dysfunction or disorder, the kit comprising primers for amplifying a target region of nuclear DNA encompassing a region of the ChAT gene, wherein said region includes one or more sites of loss-of-function mutations that are associated with a neurological dysfunction or disorder.

In some embodiments, according to the methods and kits described herein, the neurological dysfunction or disorder is selected from the group consisting of abnormal autonomic activity, functional gastrointestinal disorders, chronic pain disorders, autistic spectrum disorders, psychiatric disorders, cognitive dysfunction, and combinations thereof. In some embodiments, the individual has suffered from episodic dementia/psychosis prior to administration. In some embodiments, the individual has suffered from intestinal pseudo-obstruction prior to administration. In some embodiments, the individual has suffered from an autistic spectrum disorder prior to administration. In some embodiments, the individual has suffered an adverse reaction to an anticholinergic medication prior to administration.

In some embodiments, according to the methods and kits described herein, the individual suffers from a mitochondrial dysfunction. In some embodiments, the individual possesses homoplasmic mitochondrial DNA variants selected from the group consisting of 9070T>G in ATP6, 6253T>C in CO1, 3357C>T +2280C>T in RNR2, and combinations thereof. In some embodiments, the methods described herein further comprise sequencing mitochondrial DNA obtained from the individual. In some embodiments, the mitochondrial DNA of the individual has been sequenced without identifying heteroplasmic mitochondrial DNA variants.

In some embodiments, according to the methods and kits described herein, the loss-of-function mutation causes reduced expression of a ChAT gene product. In some embodiments, the loss-of-function mutation is in the regulatory sequence of the ChAT gene. In some embodiments, the loss-of-function mutation is in the coding sequence of the ChAT gene. In some embodiments, the loss-of-function mutation causes reduced activity of a ChAT gene product. In some embodiments, the loss-of-function mutation is or comprises a mutation selected from the group consisting of 340L>F, 510R>Q, and combinations thereof.

In some embodiments, according to the methods and kits described herein, the AChE inhibitor is selected from the group consisting of galantamine, donezepil, tacrine, rivastigmine, physostigmine, anseculin, eptastigmine, metrifonate, phenserine and pharmaceutically acceptable salts thereof. In some embodiments, the AChE inhibitor is donepezil hydrochloride.

### BRIEF DESCRIPTION OF THE DRAWING

The Figures described below, that together make up the Drawing, are for illustration purposes only, not for limitation.

FIG. 1: depicts an exemplary block diagram of a computer system 100.

FIG. 2: depicts an exemplary flow chart of a method 200 for building a database for use in selecting a medication for a patient.

FIG. 3: depicts an exemplary flow chart of a method 300 for selecting medication for a patient.

### DEFINITIONS

Acetylcholinesterase (AChE) inhibitor: As used herein, the term “AChE inhibitor” refers to any natural or synthesized compounds or molecules that influence the acetylcholine hydrolysis function of AChE. The examples of the AChE inhibitor are, but not limited to, nerve agents, organophosphorus insecticides or medicines with AChE inhibition. Generally, the AChE inhibitor is considered a nerve agent.

Associated With: The term “associated with” is used herein to describe an observed correlation between two items or events. For example, a loss-of-function mutation in ChAT may be considered to be “associated with” a particular neurological dysfunction or disorder if its presence or level correlates with a presence or level of the dysfunction or disorder.

Coding sequence: As used herein, the term “coding sequence” refers to a sequence of a nucleic acid or its complement, or a part thereof, that can be transcribed and/or translated to produce the mRNA for and/or the polypeptide or a fragment thereof. Coding sequences include exons in genomic DNA or immature primary RNA transcripts, which are joined together by the cell's biochemical machinery to provide a mature mRNA.

Dosage form: As used herein, the terms “dosage form” and “unit dosage form” refer to a physically discrete unit of a therapeutic composition for administration to a subject to be treated. Each unit dosage form contains a predetermined quantity of active agent (for example, an AChE inhibitor) calculated to produce a desired therapeutic effect when administered in accordance with a dosing regimen. It will be understood, however, that a total dosage of the active agent may be decided by an attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent (for example, an AChE inhibitor) has a recommended dosing regimen, which may involve one or more doses.

Gene: The term “gene”, as used herein, has its art understood meaning, and refers to a part of the genome specifying a macromolecular product, be it DNA for incorporation into a host genome, a functional RNA molecule or a protein, and may include regulatory sequences (e.g., promoters, enhancers, etc.) and/or intron sequences preceding (5′ non-coding sequences.

Heteroplasmic mitochondrial DNA variants: As used herein, the term “heteroplasmic mitochondrial DNA variants” refers to a mutation in mitochondrial DNA that affects a proportion of the mitochondrial DNA, while the remaining mitochondrial DNA is wild-type. In some embodiments, 0, 1, 2, 3, 4, 5, 6, 7, 8, 9, 10% or more of the mitochondrial DNA possesses the mutation.

Homoplasmic mitochondrial DNA variants: As used herein, the term “homoplasmic mitochondrial DNA variants” refers to a mutation in mitochondrial DNA that affects substantially all of the mitochondrial DNA.

Loss-of-function mutation: As used herein, the term “loss-of-function mutation” refers to a mutation that is associated with a reduction or elimination of the normal activity of a gene or gene product. Loss of activity can be due to a decrease in transcription and/or processing of the RNA, a decrease in translation, stability, transport, or activity of the gene product, or any combination thereof. In some embodiments, normal activity of a gene or gene product is reduced from 0, 1, 2, 3, 4, 5, 6, 7, 8, 9, 10 up to 100%.

Mitochondrial DNA: As used herein, the term “mitochondrial DNA” refers to the part of the genome that is located in the mitochondria of a cell.

Mitochondrial dysfunction or disorder: As used herein, the term “mitochondrial dysfunction or disorder” or also “mitochondrial disease” refers to a complex variety of symptoms. These include muscle weakness, muscle cramps, seizures, food reflux, learning disabilities, deafness, short stature, paralysis of eye muscles, diabetes, cardiac problems and stroke-like episodes, to name a few. The symptoms can range in severity from life-threatening to almost unnoticeable, sometimes taking both extremes in members of the same family. Because some people have specific subsets of these symptoms, clinical researchers have grouped those that occur together into “syndromes,” producing a bewildering array of descriptive acronyms such as MELAS (mitochondrial encephalomyopathy with lactic acidosis and stroke-like episodes) or MERFF (myoclonus epilepsy with ragged red fibers). This term also includes disorders such as Kearns-Sayre syndrome (KSS), Leigh's syndrome, maternally inherited Leigh's syndrome (MILS), Myogastrointestinal encephalomyopathy (MNGIE), Neuropathy, ataxia and retinitis pigmentosa (NARP), Friedreich's ataxia (FRDA), amyotrophic lateral sclerosis (ALS) and other motor neuron diseases, Huntington's disease, macular degeneration, epilepsy, Alzheimer's, Leber's hereditary optic neuropathy (LHON), Progressive external ophthalmoplegia (PEO), and Pearson syndrome.

Mutation: As used herein, the term “mutation” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence, or the reduction or elimination of an existing character, property, function, phenotype or trait not found in the protein encoded by the parental sequence.

Neurological dysfunction or disorders: As used herein, the term “neurological dysfunction or disorders” refers to disorders of the nervous system that result in impairment of neuronal mediated functions and includes disorders of the central nervous system (e.g., the brain, spinal cord) as well as the peripheral nervous system.

Nuclear DNA: As used herein, the term “nuclear DNA” refers to the part of the genome that is located in the nucleus of a cell.

Nucleic Acid: The terms “nucleic acid”, “nucleic acid molecule”, and “polynucleotide” each is used herein to refer to a polymers of nucleotide monomers or analogs thereof, such as deoxyribonucleic acid (DNA) and ribonucleic acid (RNA). Unless otherwise stated, the terms encompass nucleic acid-like structures with synthetic backbones, as well as amplification products. In some embodiments, nucleic acids involved in the present invention are linear nucleic acids.

Primer: The terms “primer”, as used herein, typically refers to oligonucleotides that hybridize in a sequence specific manner to a complementary nucleic acid molecule (e.g., a nucleic acid molecule comprising a target sequence). In some embodiments, a primer will comprise a region of nucleotide sequence that hybridizes to at least about 8, e.g., at least about 10, at least about 15, or about 20 to about 40 consecutive nucleotides of a target nucleic acid (i.e., will hybridize to a contiguous sequence of the target nucleic acid). In general, a primer sequence is identified as being either “complementary” (i.e., complementary to the coding or sense strand (+)), or “reverse complementary” (i.e., complementary to the anti-sense strand (−)). In some embodiments, the term “primer” may refer to an oligonucleotide that acts as a point of initiation of a template-directed synthesis using methods such as PCR (polymerase chain reaction) under appropriate conditions (e.g., in the presence of four different nucleotide triphosphates and a polymerization agent, such as DNA polymerase in an appropriate buffer solution containing any necessary reagents and at suitable temperature(s)). Such a template directed synthesis is also called “primer extension”. For example, a primer pair may be designed to amplify a region of DNA using PCR. Such a pair will include a “forward primer” and a “reverse primer” that hybridize to complementary strands of a DNA molecule and that delimit a region to be synthesized and/or amplified.

Reference: As will be understood from context, a reference sequence, sample, population, agent or individual is one that is sufficiently similar to a particular sequence, sample, population, agent or individual of interest to permit a relevant comparison (i.e., to be comparable). In some embodiments, information about a reference sample is obtained simultaneously with information about a particular sample. In some embodiments, information about a reference sample is historical. In some embodiments, information about a reference sample is stored for example in a computer-readable medium. In some embodiments, comparison of a particular sample of interest with a reference sample establishes identity with, similarity to, or difference of a particular sample of interest relative to a reference.

Regulatory Sequence: The term “regulatory sequence” is intended to include promoters, enhancers and other expression control elements (e.g., polyadenylation signals).

Risk: As will be understood from context, a “risk” of a disease, disorder or condition (e.g., a neurological dysfunction or disorder) comprises a likelihood that a particular individual will develop the disease, disorder, or condition. In some embodiments, risk is expressed as a percentage. In some embodiments, risk is from 0,1, 2, 3, 4, 5, 6, 7, 8, 9, 10 up to 100%. In some embodiments risk is expressed as a risk relative to a risk associated with a reference sample or group of reference samples. In some embodiments, a reference sample or group of reference samples have a known risk of a disease, disorder, or condition (e.g., a neurological dysfunction or disorder). In some embodiments a reference sample or group of reference samples are from individuals comparable to a particular individual. In some embodiments, relative risk is 0, 1, 2, 3, 4, 5, 6, 7, 8, 9, 10 or more.

Sample: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids extracted from a sample or obtained by subjecting a primary sample to techniques such as amplification, isolation and/or purification of certain components, etc.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” refers to an amount of a therapeutic composition (e.g., an AChE inhibitor which confers a therapeutic effect on a treated subject, at a reasonable benefit/risk ratio applicable to any medical treatment. A therapeutic effect may be objective (i.e., measurable by some test or marker) or subjective (i.e., subject gives an indication of or feels an effect). In particular, a “therapeutically effective amount” refers to an amount of a therapeutic composition effective to treat, ameliorate, or prevent a desired disease or condition, or to exhibit a detectable therapeutic or preventative effect, such as by ameliorating symptoms associated with a disease, preventing or delaying onset of a disease, and/or also lessening severity or frequency of symptoms of a disease. A therapeutically effective amount is commonly administered in a dosing regimen that may comprise multiple unit doses. A therapeutically effective amount (and/or an appropriate unit dose within an effective dosing regimen) may vary, for example, depending on route of administration, combination with other agents, etc.

Treatment: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Wild type: As used herein, the term “wild-type” refers to a typical or common form existing in nature; in some embodiments it is the most common form.

### DETAILED DESCRIPTION OF CERTAIN EMBODIMENTS

Choline O-acetyltransferase (ChAT)

ChAT is specifically expressed in cholinergic neurons. ChAT is an enzyme which catalyzes a reaction which yields the neurotransmitter acetylcholine. Although choline acetyltransferase expression has been found in both neurons and certain non-neuronal tissues, such as placenta (Schuberth, J., Biochim. Biophys. Acta, 122:470-481 (1966)) and spermatozoa (Ibanez, C. F. and Persson, H., Eur. J. Neurosci., 3:1309-1315 (1991)), the expression of this enzyme is largely limited to certain neurons.

The control of motor behavior constitutes one of the most important functions of the central nervous system. Numerous regions of the brain are involved in this process that is integrated ultimately in the motor neurons of the spinal cord, the “final common path” in the control of movement. These neurons, which lie in the ventral horn, exhibit a cholinergic phenotype and, therefore, express ChAT. ChAT is a specific marker of the cholinergic system.

ChAT has been purified, characterized, cloned and sequenced from both mouse and human sources. The ChAT protein contains 630 amino acid residues. Exemplary amino acid and nucleotide sequence from a full-length human ChAT polypeptide are shown below as SEQ IDs NO: 1 and 2.

TABLE 1
I.

| | |
|---|---|
| Human ChAT | MAAKTPSSEESGLPKLPVPPLQQTLATYLQCMRHLVSEEQFRKSQ |
| Protein Sequence | AIVQQFGAPGGLGETLQQKLLERQEKTANWVSEYWLNDMYLNNRL |
| (NCBI Reference | ALPVNSSPAVIFARQHFPGTDDQLRFAASLISGVLSYKALLDSHS |
| Sequence: | IPTDCAKGQLSGQPLCMKQYYGLFSSYRLPGHTQDTLVAQNSSIM |
| NP_001136401.1) | PEPEHVIVACCNQFFVLDVVINFRRLSEGDLFTQLRKIVKMASNE |
| | DERLPPIGLLTSDGRSEWAEARTVLVKDSTNRDSLDMIERCICLV |
| | CLDAPGGVELSDTHRALQLLHGGGYSKNGANRWYDKSLQFVVGRD |
| | GTCGVVCEHSPFDGIVLVQCTEHLLKHMTQSSRKLIRADSVSELP |
| | APRRLRWKCSPEIQGHLASSAEKLQRIVKNLDFIVYKFDNYGKTF |
| | IKKQKCSPDAFIQVALQLAFYRLHRRLVPTYESASIRRFQEGRVD |
| | NIRSATPEALAFVRAVTDHKAAVPASEKLLLLKDAIRAQTAYTVM |
| | AITGMAIDNHLLALRELARAMCKELPEMFMDETYLMSNRFVLSTS |
| | QVPTTTEMFCCYGPVVPNGYGACYNPQPETILFCISSFHSCKETS |
| | SSKFAKAVEESLIDMRDLCSLLPPTESKPLATKEKATRPSQGHQP |
| | (SEQ ID NO: 1) |
| Human ChAT | AAATGCTGAGCTAGGGGCAGGAGGCATGGGCGGGACAGTGTTCTG |
| mRNA Sequence | TGCCCCCTTCTAGAGCCTAAATTTGTTGCCCGAGTTCCTCCGGGA |
| (NCBI Reference | AGCGCTCCGGGTAGATTCTGGGGGCCGGGAGCTGAGATCCCTGGG |
| Sequence: | CGGGGAGCTGGGGAAGGGATGGGGCTGAGGACAGCGAAGAAGAGG |
| NM_001142929.1) | GGGCTTGGGGGAGGGGGGAAATGGAAGAGAGAGGAGGGAGGAGGT |
| | ACAAGAGGAAGGAGAGAAGTGCGGCCAGCTTGCTTTCTCCAGTCG |
| | GGTGGCCGCGGGGACCCGGGCGACGTCGGAGGCCCTGCCGGGAAC |
| | CCAGGCTGCAGCCCCCACCCCCGCGCTGCGACACGCCCCCCACCC |
| | CTTCCGGCTCACACCCCCGCCCACACTCCTGAGTGGTGCGGTGCA |
| | GCGTCGGCCGAGGCAGCAGAGCCGAGGAGAGCAGGTCCACACCTC |
| | TGCATCCCTGCACCAGGACTCACCAAGACGCCCATCCTGGAAAAG |
| | GTCCCCCGTAAGATGGCAGCAAAAACTCCCAGCAGTGAGGAGTCT |
| | GGGCTGCCCAAACTGCCCGTGCCCCCGCTGCAGCAGACCCTGGCC |
| | ACGTACCTGCAGTGCATGCGACACTTGGTGTCTGAGGAGCAGTTC |
| | AGGAAGAGCCAGGCCATTGTGCAGCAGTTTGGGGCCCCTGGTGGC |
| | CTCGGCGAGACCCTGCAGCAGAAACTCCTGGAGCGGCAGGAGAAG |
| | ACAGCCAACTGGGTGTCTGAGTACTGGCTGAATGACATGTATCTC |
| | AACAACCGCCTGGCCCTGCCTGTCAACTCCAGCCCTGCCGTGATC |
| | TTTGCTCGGCAGCACTTCCCTGGCACCGATGACCAGCTGAGGTTT |
| | GCAGCCAGCCTCATCTCTGGTGTACTCAGCTACAAGGCCCTGCTG |
| | GACAGCCACTCCATTCCCACTGACTGTGCCAAAGGCCAGCTGTCA |
| | GGGCAGCCCCTTTGCATGAAGCAATACTATGGGCTCTTCTCCTCC |
| | TACCGGCTCCCCGGCCATACCCAGGACACGCTGGTGGCTCAGAAC |
| | AGCAGCATCATGCCGGAGCCTGAGCACGTCATCGTAGCCTGCTGC |
| | AATCAGTTCTTTGTCTTGGATGTTGTCATTAATTTCCGCCGTCTC |
| | AGTGAGGGGGATCTGTTCACTCAGTTGAGAAAGATAGTCAAAATG |
| | GCTTCCAACGAGGACGAGCGTTTGCCTCCAATTGGCCTGCTGACG |
| | TCTGACGGGAGGAGCGAGTGGGCCGAGGCCAGGACGGTCCTCGTG |
| | AAAGACTCCACCAACCGGGACTCGCTGGACATGATTGAGCGCTGC |
| | ATCTGCCTTGTATGCCTGGACGCGCCAGGAGGCGTGGAGCTCAGC |
| | GACACCCACAGGGCACTCCAGCTCCTTCACGGCGGAGGCTACAGC |
| | AAGAACGGGGCCAATCGCTGGTACGACAAGTCCCTGCAGTTTGTG |
| | GTGGGCCGAGACGGCACCTGCGGTGTGGTGTGCGAACACTCCCCA |
| | TTCGATGGCATCGTCCTGGTGCAGTGCACTGAGCATCTGCTCAAG |
| | CACATGACGCAGAGCAGCAGGAAGCTGATCCGAGCAGACTCCGTC |
| | AGCGAGCTCCCCGCCCCCCGGAGGCTGCGGTGGAAATGCTCCCCG |
| | GAAATTCAAGGCCACTTAGCCTCCTCGGCAGAAAAACTTCAACGA |
| | ATAGTAAAGAACCTTGACTTCATTGTCTATAAGTTTGACAACTAT |
| | GGGAAAACATTCATTAAGAAGCAGAAATGCAGCCCTGATGCCTTC |
| | ATCCAGGTGGCCCTCCAGCTGGCCTTCTACAGGCTCCATCGAAGA |
| | CTGGTGCCCACCTACGAGAGCGCGTCCATCCGCCGATTCCAGGAG |
| | GGACGCGTGGACAACATCAGATCGGCCACTCCAGAGGCACTGGCT |
| | TTTGTGAGAGCCGTGACTGACCACAAGGCTGCTGTGCCAGCTTCT |
| | GAGAAGCTTCTGCTCCTGAAGGATGCCATCCGTGCCCAGACTGCA |
| | TACACAGTCATGGCCATAACAGGGATGGCCATTGACAACCACCTG |
| | CTGGCACTGCGGGAGCTGGCCCGGGCCATGTGCAAGGAGCTGCCC |
| | GAGATGTTCATGGATGAAACCTACCTGATGAGCAACCGGTTTGTC |
| | CTCTCCACTAGCCAGGTGCCCACAACCACGGAGATGTTCTGCTGC |
| | TATGGTCCTGTGGTCCCAAATGGGTATGGTGCCTGCTACAACCCC |
| | CAGCCAGAGACCATCCTTTTCTGCATCTCTAGCTTTCACAGCTGC |
| | AAAGAGACTTCTTCTAGCAAGTTTGCAAAAGCTGTGGAAGAAAGC |
| | CTCATTGACATGAGAGACCTCTGCAGTCTGCTGCCGCCTACTGAG |
| | AGCAAGCCATTGGCAACAAAGGAAAAAGCCACGAGGCCCAGCCAG |
| | GGACACCAACCTTGACTCCTGCCACTAGGTTTCACCTCCCAAACC |
| | CAGCCTCTAGAACAGCCAGACCCTGCAG (SEQ ID NO: 2) |

Reduced ChAT Function and Neurological Dysfunction or Disorders

The present invention encompasses the recognition that reduced ChAT function is associated with a risk or susceptibility to a neurological dysfunction or disorder. In some embodiments, a neurological dysfunction or disorder is any dysfunction or disorder that result in impairment of neuronal mediated functions and includes disorders of the central nervous system (e.g., the brain, spinal cord) as well as the peripheral nervous system. In some embodiments, a neurological dysfunction or disorder comprises abnormal autonomic activity. In some embodiments, a neurological dysfunction or disorder comprises functional gastrointestinal disorders (e.g., GI dysmotility, gastroesophageal reflux disease (i.e., GERD), small bowel disease, large bowel disease, irritable bowel syndrome, constipation, cyclic vomiting syndrome, etc.). In some embodiments, a neurological dysfunction or disorder comprises chronic pain disorders (e.g., migraine, abdominal pain, myalgia, etc.). In some embodiments, a neurological dysfunction or disorder comprises chronic fatigue disorders. In some embodiments, a neurological dysfunction or disorder comprises autistic spectrum disorders. In some embodiments, a neurological dysfunction or disorder comprises psychiatric disorders. In some embodiments, a neurological dysfunction or disorder comprises cognitive dysfunction and/or decline. In some embodiments, a neurological dysfunction or disorder comprises episodic encephalopathy. In some embodiments, a neurological dysfunction or disorder comprises episodic dementia/psychosis.

In some embodiments, a risk of a neurological dysfunction or disorder comprises a risk from 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference. In some embodiments, a reference comprises an average occurrence of a neurological dysfunction or disorder in a population. In some embodiments, a reference comprises a statistical occurrence of a neurological dysfunction or disorder deemed to be acceptable or unavoidable in a population by medical professionals.

ChAT Mutations

The present invention encompasses the recognition that a loss-of-function mutation in nuclear DNA that encodes ChAT can be associated with an altered risk of or suffering from a neurological dysfunction or disorder.

In some embodiments, a loss-of-function mutation is in the regulatory sequence of the ChAT gene. In some embodiments, the loss-of-function mutation is in the coding sequence of the ChAT gene. In some embodiments, the loss-of-function mutation is or comprises a mutation selected from the group consisting of 340L>F, 510R>Q, and combinations thereof.

In some embodiments, the loss-of-function mutation in nuclear DNA that encodes ChAT causes reduced expression of a ChAT gene product. In some embodiments, reduced expression of a ChAT gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a neurological dysfunction or disorder. In some embodiments, a reference is a sample from an individual known to have a wild type ChAT gene.

Methods of quantifying levels of RNA transcripts are well known in the art and include but are not limited to northern analysis, semi-quantitative reverse transcriptase PCR, quantitative reverse transcriptase PCR, and microarray analysis. These and other basic RNA transcript detection procedures are described in Ausebel et al. (1998. Current Protocols in Molecular Biology. Wiley: New York).

In some embodiments, the loss-of-function mutation causes reduced activity of a ChAT gene product. In some embodiments, reduced activity of a ChAT gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a neurological dysfunction or disorder. In some embodiments, a reference is a sample from an individual known to have a wild type ChAT gene.

Methods of quantifying activity of a ChAT gene product are well known in the art. Exemplary methods include but are not limited to using a radiometric based in vitro assay. For example, as reported by Heo Ho-Jin et al., Biosci. Biotechnol. Biochem., 67(6), 1284-1291, 2003, after choline acetyltransferase-catalyzed reaction on substrates of acetyl-CoA and choline using radiolabelled 14C-acetyl-CoA, the formed 14C-acetylcholine is extracted with tetraphenylboron (TPB). The 2 phases are separated and the radioactivity of the upper phase is measured in a liquid scintillation counter. Another assay that can be used to measure the choline acetyltransferase activity is an absorbance assay. The principle of the absorbance assay is that choline acetyltransferase activity can be determined by measuring the free Coenzyme A (CoA) formed by choline acetyltransferase reaction using 5,5′-dithio-bis(2-nitrobenzoic acid) (DTNB) reagent. DTNB reacts with free thiol groups in solution to produce 5-thio-2-nitrobenzoic acid (TNB). TNB is yellow and has absorption maximum at 412 nm. This colored TNB can then be measured by absorbancy at 405 nm.

Diagnosis of Neurological Dysfunctions or Disorders

In some embodiments, the present invention provides methods of classifying an individual at risk of or suffering from a neurological dysfunction or disorder. In general, such methods comprise obtaining a sample of nuclear DNA from the individual; processing the sample to determine whether the individual possesses a mutation in nuclear DNA that encodes ChAT; and classifying the individual as one that does or does not possess a mutation in nuclear DNA that encodes ChAT.

In some embodiments, an individual at risk of or suffering from a neurological dysfunction or disorder is a non-human animal. In some embodiments, a non-human animal is a mouse. In some embodiments, a non-human animal is a rat. In some embodiments, a non-human animal is a dog. In some embodiments, a non-human animal is a non-human primate. In some embodiments, an individual is a human. In some embodiments, a sample is obtained from an individual harboring a ChAT mutation. In some embodiments, a sample is obtained from an individual harboring a loss-of-function mutation in nuclear DNA that encodes ChAT described herein.

In some embodiments, an individual at risk of or suffering from a neurological dysfunction or disorder suffers from a mitochondrial dysfunction or disorder. Many neurological dysfunctions and disorders are mitochondria driven and share common genomic malfunctions with mitochondrial dysfunctions and disorders. Mitochondrial dysfunction or disorders are degenerative diseases due to various mechanisms such as abnormality of mitochondrial DNA (deletion, point mutation, and duplication), abnormality of cellular DNA encoding mitochondrial enzymes or complex polymeric mitochondrial components, or can be induced by toxic substances or pharmaceutical products. When mitochondria-associated genes are damaged because of these reasons, various biochemical abnormalities occur.

In some embodiments, an individual possessing a mutation in their nuclear DNA that encodes ChAT does not possesses heteroplasmic mitochondrial DNA variants. In some embodiments, an individual possessing a mutation in their nuclear DNA that encodes ChAT possesses one or more homoplasmic mitochondrial DNA variants. In some embodiments, homoplasmic DNA variants are selected from the group consisting of 9070T>G in ATP6, 6253T>C in CO1, 3357C>T+2280C>T in RNR2, and combinations thereof. Methods for sequencing mitochondrial DNA are well known in the art.

In some embodiments, a sample is any sample comprising ChAT nuclear DNA. In some embodiments, a sample comprises cells from which nuclear DNA (e.g., not mitochondrial DNA) is or can be obtained. In some embodiments, a sample comprises cells from which mitochondrial DNA is or can be obtained. In some embodiments, a sample comprises isolated nucleic acids. In some embodiments, a sample comprises genomic DNA. In some embodiments, a sample comprises human genomic DNA.

In some embodiments, processing comprises processing a sample to detect a sequence of nuclear DNA that encodes ChAT. In some embodiments, processing a sample comprises amplifying a target nucleic acid region of human genomic DNA encompassing a region that encodes the ChAT polypeptide, wherein said region includes one or more sites of loss-of-function mutations that are associated with a neurological dysfunction or disorder. In some embodiments, amplifying comprises contacting the human genomic DNA with a 5′ primer under conditions such that hybridization and extension of the target nucleic acid region occur in a forward direction. In some embodiments, amplifying further comprises contacting the human genomic DNA with a 3′ primer under conditions such that hybridization and extension of the target nucleic acid region occur in a reverse direction.

Nucleic acid amplification methods are well known in the art and include, but are not limited to, the Polymerase Chain Reaction (or PCR, described, for example, in U.S. Pat. Nos. 4,683,195, 4,683,202 and 4,889,818, each of which is incorporated herein by reference in its entirety). In its simplest form, PCR is an in vitro method for the enzymatic synthesis of specific DNA sequences, using two primers that hybridize to opposite strands and flank the region of interest in the target DNA. A plurality of reaction cycles, each cycle comprising: a denaturation step, an annealing step, and a polymerization step, results in the exponential accumulation of a specific DNA fragment. The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq) which are available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs.

In some embodiments, the one or more sites of loss-of-function mutations correspond to amino acids 340 and/or 510 of a ChAT gene product. In some embodiments, the loss-of-function mutations are selected from the group consisting of 340L>F, 510R>Q, and combinations thereof.

In some embodiments, a first amplification step amplifies a region of a target gene. In some embodiments the amplification product is less than about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 250, 225, 200, 175 or 150 nucleotides long.

In some embodiments, processing a sample comprises genotyping a nucleic acid (e.g., an amplified nucleic acid) using techniques described herein. In some embodiments, an individual is classified as at risk of or suffering from a neurological dysfunction or disorder if they are determined by genotyping to have one or more mutant alleles. In some embodiments, mutant alleles encode a ChAT mutation described herein whose presence correlates with incidence and/or risk of a neurological dysfunction or disorder.

Common genotyping methods are known in the art and include, but are not limited to, sequencing, quantitative PCR, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

In some embodiments genotyping nuclear DNA that encodes ChAT comprises sequencing the amplified DNA. In some embodiments, any of a variety of sequencing reactions known in the art can be used to directly sequence at least a portion of amplified DNA. Exemplary sequencing reactions include those based on techniques developed by Maxam and Gilbert, Proc. Natl. Acad. Sci USA, 74:560 (1977) or Sanger, Proc. Nat. Acad. Sci 74:5463 (1977). It is also contemplated that any of a variety of automated sequencing procedures may be utilized when performing the subject assays, e.g., see Venter et al., Science, 291:1304-1351 (2001); Lander et al., Nature, 409:860-921 (2001), including sequencing by mass spectrometry, e.g., see U.S. Pat. No. 5,547,835 and PCT Patent Publication No. WO 94/16101 and WO 94/21822; U.S. Pat. No. 5,605,798 and PCT Patent Application No. PCT/US96/03651; Cohen et al., Adv. Chromatogr. 36:127-162 (1996); and Griffin et al., Appl. Biochem. Biotechnol. 38:147-159 (1993). It will be evident to one skilled in the art that, for some embodiments, the occurrence of only one, two or three of the nucleic acid bases need be determined in the sequencing reaction. Yet other sequencing methods are disclosed, e.g., in U.S. Pat. Nos. 5,580,732; 5,571,676; 4,863,849; 5,302,509; PCT Patent Application Nos. WO 91/06678 and WO 93/21340; Canard et al., Gene 148:1-6 (1994); Metzker et al., Nucleic Acids Research 22:4259-4267 (1994) and U.S. Pat. Nos. 5,740,341 and 6,306,597. In some embodiments, sequencing reactions comprise deep sequencing.

In some embodiments, genotyping nuclear DNA that encodes ChAT comprises hybridizing a nucleic acid detection probe to the amplified DNA, wherein the nucleic acid detection probe comprises sequence that is complimentary to the sequence of the at least one mutation. In some embodiments, hybridization of the nucleic acid detection probe to the amplified human genomic DNA is detected by quantitative PCR. “Quantitative” PCR which are also referred to as “real-time PCR” and “real-time RT-PCR,” respectively, involves detecting PCR products via a probe that provides a signal (typically a fluorescent signal) that is related to the amount of amplified product in the sample. Examples of commonly used probes used in quantitative include the following probes: TAQMAN® probes, Molecular Beacons probes, SCORPION® probes, and SYBR® Green probes. Briefly, TAQMAN® probes, Molecular Beacons, and SCORPION® probes each have a fluorescent reporter dye (also called a “fluor”) attached on or around the 5′ end of the probes and a quencher moiety attached on or around the 3′ end of the probes. In the unhybridized state, the proximity of the fluor and the quench molecules prevents the detection of fluorescent signal from the probe. During PCR, when the polymerase replicates a template on which a probe is bound, the 5′-nuclease activity of the polymerase cleaves the probe at a site between the fluor and quencher thus, increasing fluorescence with each replication cycle. SYBR® Green probes bind double-stranded DNA and upon excitation emit light; thus as PCR product accumulates, fluorescence increases.

In some embodiments, the nucleic acid detection probe detect nucleic acids that encode a 340L>F mutation of ChAT. In some embodiments, the nucleic acid detection probe detect nucleic acids that encode a 510R>Q mutation of ChAT.

In some embodiments genotyping nuclear DNA that encodes ChAT comprises a primer extension reaction. Several such methods have been described in the patent and scientific literature and include the “Genetic Bit Analysis” method (W)92/15712) and the ligase/polymerase mediated genetic bit analysis (U.S. Pat. No. 5,679,524). Related methods are disclosed in WO91/02087, WO90/09455, WO95/17676, U.S. Pat. Nos. 5,302,509, and 5,945,283. In some embodiments a primer extension reaction comprises contacting the amplified nucleic acid with one or more primers which specifically hybridize to a region of the isolated nucleic acid containing a mutation, and amplifying the hybridized amplified nucleic acid to detect the nucleotide present at the position of interest. In some embodiments detecting the presence or absence of an amplification product (assays can be designed so that hybridization and/or amplification will only occur if a particular mutation is present or absent).

Acetylcholinesterase (AChE) Inhibitors

The present invention encompasses the recognition that inhibition of AChE activity, wherein nuclear DNA of the individual that encodes ChAT includes a loss-of function mutation, represents an effective therapy for neurological dysfunctions or disorders. In some embodiments, the current invention provides methods of treating or reducing risk for a neurological dysfunction or disorder comprising administering to a subject one or more AChE inhibitors. In certain embodiments, the methods comprise administering to the individual a therapeutically effective amount of an AChE inhibitor, wherein nuclear DNA of the individual that encodes ChAT includes a loss-of function mutation.

In some embodiments, classifying the individual as one that does or does not possess a mutation in nuclear DNA that encodes ChAT according to the methods described herein further comprises providing the individual or a physician treating the individual with information regarding the mutation. In some embodiments, the information references a correlation between the mutation and the potential benefits of therapy with an AChE inhibitor.

In some embodiments, the invention described herein comprises methods of aiding in the selection of a therapy for an individual at risk of or suffering from a neurological dysfunction or disorder, the method comprising obtaining a sample of nuclear DNA from the individual, processing the sample to determine whether the individual possesses a loss-of-function mutation in nuclear DNA that encodes ChAT, and classifying the individual as one that could benefit from therapy with an AChE inhibitor if the step of processing determines that the individual possesses a loss-of-function mutation in nuclear DNA that encodes ChAT using techniques described herein.

A variety of AChE inhibitors may be used in methods of the present disclosure. Representative AChE inhibitors include galantamine, donezepil, tacrine, rivastigmine, physostigmine, anseculin, eptastigmine, metrifonate, phenserine and pharmaceutically acceptable salts thereof. These and other representative AChE inhibitors including exemplary dosages are set forth in U.S. Pat. Nos. 4,914,102; 5,100,901; 5,102,891; 5,166,181; 5,187,165; 5,288,758; 5,302,593; 5,300,517; 5,338,548; 5,364,864; 5,389,629; 5,391,553; 5,455,245; 5,574,046; 5,602,176; 5,622,976; 5,663,448; 5,693,668 and 5,744,476; European Patent Application Nos. 268,871; 298,202; 409,676; 477,903 and 703,901; and PCT WO 93/13100; 93/16690; 96/40682; 97/19059 and 97/38993, the disclosures of which are incorporated herein by reference in their entirety. In certain embodiments, the AChE inhibitor is donepezil hydrochloride.

In accordance with the methods of the invention, an AChE inhibitor can be administered to a subject alone, or as a component of a composition or medicament (e.g., in the manufacture of a medicament for the prevention or treatment of a neurological dysfunction or disorder), as described herein. The compositions can be formulated with a physiologically acceptable carrier or excipient to prepare a pharmaceutical composition. The carrier and composition can be sterile. The formulation should suit the mode of administration. Methods of formulating compositions are known in the art (see, e.g., Remington's Pharmaceuticals Sciences, 17^{th} Edition, Mack Publishing Co., (Alfonso R. Gennaro, editor) (1989)). Suitable pharmaceutically acceptable carriers are known in the art.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulations can include standard carriers such as pharmaceutical grades of mannitol, lactose, starch, magnesium stearate, polyvinyl pyrollidone, sodium saccharine, cellulose, magnesium carbonate, etc.

An AChE inhibitor described herein (or a composition or medicament containing an agent described herein) is administered by any appropriate route. In some embodiments, an AChE inhibitor is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, thigh region, abdominal region, gluteal region, or scapular region. In some embodiments, an AChE inhibitor is administered intravenously. In some embodiments, an AChE inhibitor is administered orally. In other embodiments, an AChE inhibitor is administered by direct administration to a target tissue, such as heart or muscle (e.g., intramuscular), tumor (intratumorallly), nervous system (e.g., direct injection into the brain; intraventricularly; intrathecally). Alternatively, an AChE inhibitor (or a composition or medicament containing an agent) can be administered by inhalation, parenterally, intradermally, transdermally, or transmucosally (e.g., orally or nasally). More than one route can be used concurrently, if desired.

In various embodiments, an AChE inhibitor is administered at a therapeutically effective amount. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating the underlying disease or condition). In some particular embodiments, appropriate doses or amounts to be administered may be extrapolated from dose-response curves derived from in vitro or animal model test systems.

In some embodiments, a composition is administered in a therapeutically effective amount and/or according to a dosing regimen that is correlated with a particular desired outcome (e.g., with treating or reducing risk for a neurological dysfunction or disorder).

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, or combinations thereof).

In some embodiments, a provided composition is provided as a pharmaceutical formulation. In some embodiments, a pharmaceutical formulation is or comprises a unit dose amount for administration in accordance with a dosing regimen correlated with achievement of the reduced incidence or risk of a neurological dysfunction or disorder.

In some embodiments, a formulation comprising an AChE inhibitor described herein is administered as a single dose. In some embodiments, a formulation comprising an AChE inhibitor described herein is administered at regular intervals. Administration at an “interval,” as used herein, indicates that the therapeutically effective amount is administered periodically (as distinguished from a one-time dose).

In some embodiments, a formulation comprising an AChE inhibitor described herein is administered at regular intervals indefinitely. In some embodiments, a formulation comprising an AChE inhibitor described herein is administered at regular intervals for a defined period.

Kits

In some embodiments, the present invention provides kits comprising materials useful for the amplification and detection or sequencing of the nuclear DNA that encompasses part or all of the ChAT gene product according to methods described herein. The inventive kits may be used by diagnostic laboratories, experimental laboratories, or practitioners. In some embodiments, the present disclosure provides kits further comprising materials useful for treating a neurological dysfunction or disorder. In some embodiments, the materials useful for treating the neurological dysfunction or disorder are AChE inhibitors.

Materials and reagents useful for the detection or sequencing of the nuclear DNA that encompasses part or all of the ChAT gene product according to the present disclosure may be assembled together in a kit. In some embodiments, an inventive kit comprises at least one inventive primer set, and optionally, amplification reaction reagents. In some embodiments, a kit comprises reagents which render the procedure specific. In some embodiments, the kit comprises nucleic detection probes. Thus, a kit intended to be used for the detection of a particular loss-of-function mutation (e.g., 340L>F or 510R>Q) preferably comprises primer sets and/or probes described herein that can be used to amplify and/or detect a particular ChAT target sequence of interest. A kit intended to be used for the multiplex detection of a plurality of ChAT target preferably comprises a plurality of primer sets and/or probes (optionally in separate containers) described herein that can be used to amplify and/or detect ChAT target sequences described herein.

Suitable amplification reaction reagents that can be included in an inventive kit include, for example, one or more of: buffers; enzymes having polymerase activity; enzyme cofactors such as magnesium or manganese; salts; nicotinamide adenide dinuclease (NAD); and deoxynucleoside triphosphates (dNTPs) such as, for example, deoxyadenosine triphospate; deoxyguanosine triphosphate, deoxycytidine triphosphate and deoxythymidine triphosphate, biotinylated dNTPs, suitable for carrying out the amplification reactions.

Depending on the procedure, the kit may further comprise one or more of: wash buffers and/or reagents, hybridization buffers and/or reagents, labeling buffers and/or reagents, and detection means. The buffers and/or reagents included in a kit are preferably optimized for the particular amplification/detection technique for which the kit is intended. Protocols for using these buffers and reagents for performing different steps of the procedure may also be included in the kit.

Furthermore, the kits may be provided with an internal control as a check on the amplification procedure and to prevent occurrence of false negative test results due to failures in the amplification procedure. An optimal control sequence is selected in such a way that it will not compete with the target nucleic acid sequence in the amplification reaction (as described above).

Kits may also contain reagents for the isolation of nucleic acids from biological specimen prior to amplification.

The reagents may be supplied in a solid (e.g., lyophilized) or liquid form. The kits of the present disclosure optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the amplification/detection assay may also be provided. The individual containers of the kit are preferably maintained in close confinement for commercial sale.

The kit may also comprise instructions for using the amplification reaction reagents, primer sets, primer/probe sets and/or AChE inhibitor according to the present disclosure. Instructions for using the kit according to one or more methods of the present disclosure may comprise instructions for processing the biological sample, extracting nucleic acid molecules, and/or performing the test; instructions for interpreting the results as well as a notice in the form prescribed by a governmental agency (e.g., FDA) regulating the manufacture, use or sale of pharmaceuticals or biological products.

Computer Systems

Methods described herein can be implemented in a computer system having a processor that executes specific instructions in a computer program. The computer system may be arranged to output a medication profile based on receiving an individual's genotype (e.g., ChAT polymorphism(s) and/or mitochondrial DNA variants). It is to be understood that an individual's genotypic information may be gathered and/or received in the form of amino acid and/or a nucleotide data. Particularly, the computer program may include instructions for the system to select the most appropriate medication (e.g., an AChE inhibitor or a particular AChE inhibitor) for an individual.

In some embodiments, the computer program may be configured such that the computer system can identify the genotype based on received data and provide a preliminary identification of the universe of possible medications. The system may be able to rank-order the identified medications based on specific co-factors in the algorithmic equation. The system may be able to adjust the rank ordering based on the individual's genotype. The system may be able to adjust the rank ordering based on clinical responses, such as by family members of the individual.

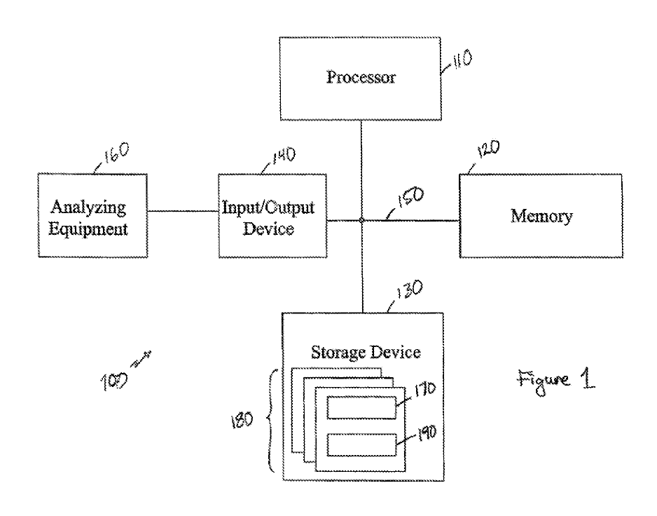

FIG. 1 is a block diagram of a computer system 100 that can be used in the operations described above, according to one embodiment. The system 100 includes a processor 110, a memory 120, a storage device 130 and an input/output device 140. Each of the components 110, 120, 130 and 140 are interconnected using a system bus 150. The system may include analyzing equipment 160 for determining the individual's genotype.

The processor 110 is capable of processing instructions for execution within the system 100. In one embodiment, the processor 110 is a single-threaded processor. In another embodiment, the processor 110 is a multi-threaded processor. The processor 110 is capable of processing instructions stored in the memory 120 or on the storage device 130, including for receiving or sending information through the input/output device 140.

The memory 120 stores information within the system 100. In one embodiment, the memory 120 is a computer-readable medium. In one embodiment, the memory 120 is a volatile memory unit. In another embodiment, the memory 120 is a non-volatile memory unit.

The storage device 130 is capable of providing mass storage for the system 100. In one embodiment, the storage device 130 is a computer-readable medium. In various different embodiments, the storage device 130 may be a floppy disk device, a hard disk device, an optical disk device, or a tape device.

The input/output device 140 provides input/output operations for the system 100. In one embodiment, the input/output device 140 includes a keyboard and/or pointing device. In one embodiment, the input/output device 140 includes a display unit for displaying graphical user interfaces.

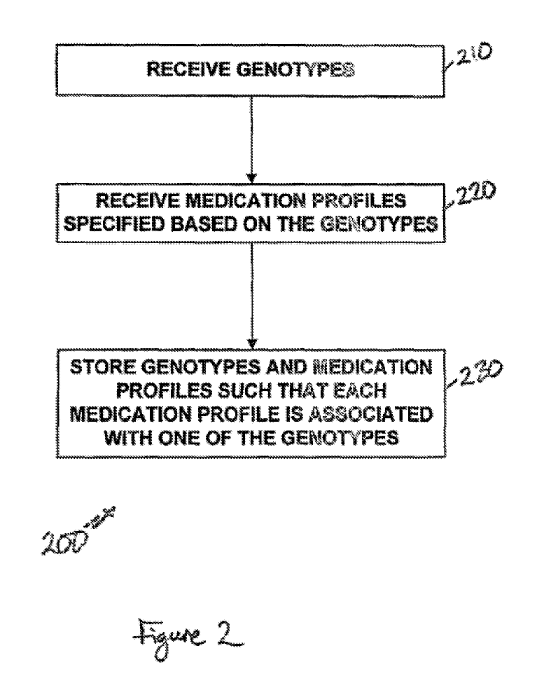

The system 100 can be used to build a database. FIG. 2 shows a flow chart of a method 200 for building a database for use in selecting a medication for an individual. Preferably, the method 200 is performed in the system 100. For example, a computer program product can include instructions that cause the processor 110 to perform the steps of the method 200. The method 200 includes the following steps.

Receiving, in step 210, a plurality of genotypes 170 for ChAT and/or mitochondrial variants. A computer program in the system 100 may include instructions for presenting a suitable graphical user interface on input/output device 140, and the graphical user interface may prompt the user to enter the genotypes 170 using the input/output device 140, such as a keyboard.

Receiving, in step 220, a plurality of medication profiles 180. The medication profiles 180 are specified based on the genotypes 170. The user may enter the medication profiles 180 using the input/output device 140, such as a keyboard. For example, the medication profile 180 may include information 190 regarding at least one medication.

Storing, in step 230, the received genotypes 170 and the medication profiles 180 such that each medication profile 180 is associated with one of the genotypes 170. The system 100 may store the medication profiles 180 and the genotypes 170 in the storage device 130. For example, when the storing is complete, the system 100 can identity a particular one of the medication profiles 180 that is associated with a specific genotype 170. Having identified the medication profile 180, the system 100 can access the information 190 contained within the identified medication profile 180, as will be described in the following example.

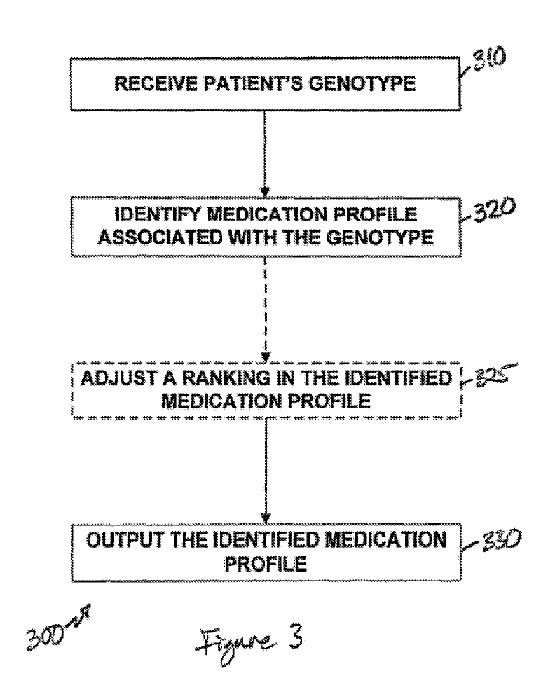

The system 100 may be used for selecting a medication. FIG. 3 shows a flow chart of a method 300 of selecting a medication for an individual. Preferably, the method 300 is performed in the system 100. For example, a computer program product can include instructions that cause the processor 110 to perform the steps of the method 300. The method 300 includes the following steps.

Receiving, in step 310, an individual's genotype for ChAT and/or mitochondrial variants. The genotype may be entered by a user via input/output device 140. For example, the user may obtain the individual's genotype for ChAT and/or mitochondrial variants using the analyzing equipment 160 (which may or may not be connected to the system 100). The user may type the individual's genotype on input/output device 140, such as a keyboard, for receipt by the system 100.

The genotype may be received directly from the analyzing equipment 160. For example, analyzing equipment 160 may include a processor and suitable software such that it can communicate over a network. The system 100 may be connected to the analyzing equipment 160 through input/output device 140, such as a network adapter, and directly receive the individual's genotype.

Identifying, in step 320, one of the medication profiles 180 that is associated with the individual's genotype. For example, the system 100 may perform a database search in the storage device 130. Particularly, the system 100 may access the genotype 170 for individual medication profiles 180 until a match is found. Optional step 325 will be described below.

Outputting, in step 330, the identified medication profile 180 in response to receiving the individual's genotype. The system may output the identified medication profile 180 through input/output device 140. For example, the identified medication profile may be printed or displayed in a suitable graphical user interface on a display device. As another example, the system 100 may transmit the identified medication profile over a network, such as a local area network or the Internet, to which the input/output device 140 is connected.

The medication profiles 180 can be created such that there is flexibility in how the system 100 outputs them. For example, the information 190 in one or more of the edication profiles 180 may include a ranking of several medications. The program may include instructions for applying rules to the received individual's genotype and adjust the ranking accordingly. In such implementations, the method 300 may include optional step 325 of adjusting the ranking before outputting the identified medication profile. For example, the system 100 may receive genotypic data carried by the individual (optionally in the same way the individual's genotype was received) and adjust the ranking accordingly in step 325. As another example, step 325 may involve adjusting the ranking based on a clinical response. The clinical response may be received by the system 100 in the same way as the individual's genotype. For example, the ranking can be adjusted based on a clinical response by a member of the individual's family.

The medication profiles 180 may be updated as necessary. For example, the introduction of a new medication on the market may prompt a revision of one or more existing medication profiles. A new medication may also be the basis for creating a new medication profile. The adjustment or creation of medication profiles may be done substantially as described above.

The medication profiles 180 may be used for medication selection in the same system where they were created, or in a different system. That is, the system 100 may first be used for building a database of the medication profiles 180, and the system 100 may thereafter be used to select a medication profile for the genotype of a specific individual. As another example, one or more medication profiles 180 may be transmitted within a computer readable medium such as a global computer network for remote processing according to the invention.

### Exemplification

While evaluating patients with suspected mitochondrial disease, it is commonplace to find a family that demonstrates apparent maternal inheritance of relatively-mild disease while one individual is far more severely affected, yet no heteroplasmic mutation is identified. This suggests the existence of nuclear-encoded modifiers that intensify the phenotype. In 9 such cases evaluated at our center, the 1,092 nuclear genes that code for mitochondrial-located proteins were sequenced by NextGen methodology (nucSEEK™). In 3 of the 9, a single variant was found in the ChAT gene, 340L>F in 2 patients and 510R>Q in the other, that are predicted to be deleterious based on very-high evolutionary conservation through vertebrates, as well as by prediction of 5 computer algorithms of protein function.

ChAT encodes for choline O-acetyltransferase, the enzyme that synthesizes acetylcholine, which is of special importance in the parasympathetic nervous system, acting as both the pre-synaptic and post-synaptic neurotransmitter. The phenotype in all 3 patients paralleled that of the known adverse effects of anticholinergic medications, including episodic dementia/psychosis and findings consistent with parasympathetic down-regulation such as intestinal pseudo-obstruction. Autistic spectrum disorders were present to various degrees in the 2 male patients and in the sister of the female patient. Of particular interest, prior to sequencing, 2/2 patients had severe reactions to medications with anticholinergic effects, and both had clinical improvement with propranolol, which dampens opposing sympathetic activity. Treatment with an AChE inhibitor (Aricept™) in two patients resulted in substantial anecdotal benefit in which one, for the first time, was able to engage in meaningful full sentences and social exchanges. The second patient has experienced the reversal of cognitive decline.

The ChAT gene variants are not rare (0.51% and 0.11% of the population), thus they likely cause disease only in the context of another genetic factor(s). All 3 families had pedigrees that are very-highly consistent with apparent maternal inheritance, but full mtDNA sequencing by NextGen failed to reveal heteroplasmy. Homoplasmic mtDNA variants of unclear significance were found in each patient: 9070T>G in ATP6, 6253T>C in CO1, and 3357C>T+2280C>T in RNR2. We suspect that relatively-mild mitochondrial dysfunction in these families is inherited on the mitochondrial DNA, but that disease only becomes substantial when modified by autonomic dysfunction due to a CHAT mutation. This type of phenomenon may explain some of the more unusual aspects of mitochondrial disease, including extreme phenotypic variability without heteroplasmy, pronounced autonomic effects, and unusual response to medications.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A method of treating an individual at risk of or suffering from a neurological dysfunction or disorder, the method comprising administering to the individual a therapeutically effective amount of an acetylcholinesterase (AChE) inhibitor, wherein nuclear DNA of the individual that encodes choline O-acetyltransferase (ChAT) includes a loss-of-function mutation.

2. A method of treating an individual at risk of or suffering from a neurological dysfunction or disorder, the method comprising administering to the individual a therapeutically effective amount of an acetylcholinesterase (AChE) inhibitor, wherein, prior to administration, the individual has been determined to possess a loss-of-function mutation in nuclear DNA that encodes choline O-acetyltransferase (ChAT).

3. A method of treating an individual at risk of or suffering from a neurological dysfunction or disorder, the method comprising determining that the individual possesses a loss-of-function mutation in nuclear DNA that encodes choline O-acetyltransferase (ChAT) and administering to the individual a therapeutically effective amount of an acetylcholinesterase (AChE) inhibitor.

4. The method of claim 2, wherein the neurological dysfunction or disorder is selected from the group consisting of abnormal autonomic activity, functional gastrointestinal disorders, chronic pain disorders, autistic spectrum disorders, psychiatric disorders, cognitive dysfunction, and combinations thereof.

5. The method of claim 4, wherein the neurological dysfunction or disorder is selected from the group consisting of an abnormal autonomic activity, a functional gastrointestinal disorder, an autistic spectrum disorder, a psychiatric disorder, and a cognitive dysfunction.

6-10. (canceled)

11. The method of claim 2, wherein, prior to administration, the individual has suffered from a condition selected from the group consisting of episodic dementia/psychosis, intestinal pseudo-obstruction, an autistic spectrum disorder, and an adverse reaction to an anticholinergic medication.

12-14. (canceled)

15. The method of claim 2, wherein the individual suffers from a mitochondrial dysfunction.

16. The method of claim 2, wherein the individual possesses homoplasmic mitochondrial DNA variants selected from the group consisting of 9070T>G in ATP6, 6253T>C in CO1, 3357C>T+2280C>T in RNR2, and combinations thereof.

17. The method of claim 2, further comprising sequencing mitochondrial DNA obtained from the individual.

18. The method of claim 2, wherein mitochondrial DNA of the individual has been sequenced without identifying heteroplasmic mitochondrial DNA variants.

19. The method of claim 2, wherein the loss-of-function mutation causes reduced expression of a ChAT gene product.

20. The method of claim 2, wherein the loss-of-function mutation is in the regulatory sequence of the ChAT gene.

21. The method of claim 2, wherein the loss-of-function mutation is in the coding sequence of the ChAT gene.

22. The method of claim 2, wherein the loss-of-function mutation causes reduced activity of a ChAT gene product.

23. The method of claim 2, wherein the loss-of-function mutation is or comprises a mutation selected from the group consisting of 340L>F, 510R>Q, and combinations thereof.

24. The method of claim 2, wherein the AChE inhibitor is selected from the group consisting of galantamine, donezepil, tacrine, rivastigmine, physostigmine, anseculin, eptastigmine, metrifonate, phenserine and pharmaceutically acceptable salts thereof.

25. The method of claim 24, wherein the AChE inhibitor is donepezil hydrochloride.

26-111. (canceled)

112. The method of claim 3, wherein determining that the individual possesses a loss-of-function mutation in nuclear DNA that encodes choline O-acetyltransferase (ChAT) comprises requesting sequencing of at least a portion of nuclear DNA that encodes ChAT.

113. The method of claim 3, wherein determining that the individual possesses a loss-of-function mutation in nuclear DNA that encodes choline O-acetyltransferase (ChAT) comprises sequencing at least a portion of nuclear DNA that encodes ChAT.

114. The method of claim 3, wherein determining that the individual possesses a loss-of-function mutation in nuclear DNA that encodes choline O-acetyltransferase (ChAT) comprises requesting genotyping of at least a portion of nuclear DNA that encodes ChAT.

115. The method of claim 3, wherein determining that the individual possesses a loss-of-function mutation in nuclear DNA that encodes choline O-acetyltransferase (ChAT) comprises genotyping at least a portion of nuclear DNA that encodes ChAT.


SEQUENCE LISTING (2 sequences)

SEQ ID NO: 1 - protein, 630 aa, Homo sapiens
MAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHMTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP

SEQ ID NO: 2 - DNA, 2458 bp, Homo sapiens
aaatgctgagctaggggcaggaggcatgggcgggacagtgttctgtgcccccttctagagcctaaatttgttgcccgagttcctccgggaagcgctccgggtagattctgggggccgggagctgagatccctgggcggggagctggggaagggatggggctgaggacagcgaagaagagggggcttgggggaggggggaaatggaagagagaggagggaggaggtacaagaggaaggagagaagtgcggccagcttgctttctccagtcgggtggccgcggggacccgggcgacgtcggaggccctgccgggaacccaggctgcagcccccacccccgcgctgcgacacgccccccaccccttccggctcacacccccgcccacactcctgagtggtgcggtgcagcgtcggccgaggcagcagagccgaggagagcaggtccacacctctgcatccctgcaccaggactcaccaagacgcccatcctggaaaaggtcccccgtaagatggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaggccagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacgcgccaggaggcgtggagctcagcgacacccacagggcactccagctccttcacggcggaggctacagcaagaacggggccaatcgctggtacgacaagtccctgcagtttgtggtgggccgagacggcacctgcggtgtggtgtgcgaacactccccattcgatggcatcgtcctggtgcagtgcactgagcatctgctcaagcacatgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccatcgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaaccttgactcctgccactaggtttcacctcccaaacccagcctctagaacagccagaccctgcag